(19) United States
(12) Patent Application Publication
(10) Pub. No.: US 2022/0378325 A1
(43) Pub. Date: Dec. 1, 2022

(54) Distributed Radar System With Active Tags For Precise Geolocation

(71) Applicant: Metawave Corporation, Carlsbad, CA (US)

(72) Inventor: Abdullah Ahsan ZAIDI, San Diego, CA (US)

(21) Appl. No.: 17/824,873

(22) Filed: May 25, 2022

Related U.S. Application Data
(60) Provisional application No. 63/193,021, filed on May 25, 2021.

(51) Int. Cl. A61B 5/107; G01S 13/84; A61B 5/05

(52) U.S. Cl. CPC A61B 5/1072; G01S 13/84; A61B 5/05

(57) ABSTRACT

In the present disclosure, a radar system is configured to interact with beacons that shift the phase of a received radar transmission to generate a phase shifted response signal. Phase shifters are designed to assign specific frequency responses to identify target locations. The radar module transmits at a modulated signal at first frequency, each beacon receives the radar transmission, phase shifts the signal and returns the phase shifted signal. Where two or more beacons are used, each will apply a different phase shift to the received radar transmission, wherein the frequency identifies the specific beacons. In a radar system, the modulated transmission signal is compared to the returned phase shifted signal to determine a frequency difference between the two signals.

### CROSS REFERENCE—CLAIM OF PRIORITY

This application claims priority from U.S. Provisional Application No. 63/193,021, titled “Distributed Radar System With Active Tags For Precise Geolocation,” filed on May 25, 2021, and incorporated herein by reference in its entirety.

### BACKGROUND

As visualization and navigation system capabilities expand, there are many new and exciting applications for their use. Particularly in the medical field, cameras and wireless communication provides tools for surgeons and others to gain information for procedures involving small anatomical regions, giving the physicians additional visualization and accuracy in their work. Robotic applications in medicine is one of many critical applications of new technologies.

### BRIEF DESCRIPTION OF THE DRAWINGS

The present application may be more fully appreciated in connection with the following detailed description taken in conjunction with the accompanying drawings, which are not drawn to scale, in which like reference characters refer to like parts throughout, and in which:

FIG. 1 illustrates an example deployment of a radar system in a medical environment, in accordance with one or more implementations of the subject technology;

FIGS. 2A-2C illustrate an example anatomical situation wherein a radar system may be implemented in accordance with one or more implementations of the subject technology;

FIG. 3 illustrates an example implementation of a radar system, in accordance with one or more implementations of the subject technology;

FIG. 4 illustrates example data acquired via a radar system, in accordance with one or more implementations of the subject technology;

FIG. 5 illustrates a schematic diagram of a radar system in accordance with one or more implementations of the subject technology;

FIG. 6 illustrates components of an example beacon, in accordance with one or more implementations of the subject technology; and

FIG. 7 illustrate a flowchart for operating a radar system, in accordance with one or more implementations of the subject technology.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology is not limited to the specific details set forth herein and may be practiced using one or more implementations. In one or more instances, structures and components are shown in block diagram form in order to avoid obscuring the concepts of the subject technology. In other instances, well-known methods and structures may not be described in detail to avoid unnecessarily obscuring the description of the examples. Also, the examples may be used in combination with each other.

The present disclosure relates to applications using a radar system for use in the field of medicine. Specifically, the technology disclosed herein relates to a radar system that is configured for use with two or more beacons for precise geotagging during a surgery (surgical operation) that requires accuracy and reliability. As used herein, a beacon, in different embodiments, can be a radio frequency integrated circuit (RFIC) based phase shifter modules. In some implementations, the beacon can include additional passive or active components, e.g., Bluetooth low energy beacons, a general communication device that is configured to receive and transmit signals at 5G, WiFi 6, or any next generation millimeter wave communication protocols. The radar system may utilize radar and phase shifter modules in various applications. Although specific applications disclosed herein are related to or for medical system, alternate applications may include a wide variety of systems requiring accuracy and reliability.

In the present disclosure, a radar system is configured to interact with a plurality of radar modules (e.g., navigation modules). In accordance with various embodiments, the radar modules each can include one or more phase shifters to adjust frequency of the received signal. A phase shifter can be configured as a beacon (e.g., a beacon unit) designed to assign specific frequency responses from one or more radar modules to identify a target location to which the beacon is adhered (e.g., attached removably, implanted or disposed permanently). As disclosed herein, a radar module can be configured to transmit a modulated signal at a first frequency, where each beacon receives the radar transmission, phase shifts the signal and returns the phase shifted signal. Where two or more (navigation) beacons are used, each will apply a different phase shift to the received radar transmission, wherein the frequency identifies the individual beacon. In a radar system, the modulated transmission signal is compared to the returned phase shifted signal to determine a frequency difference between the two signals. This is referred to as the Doppler frequency.

In various embodiments, a beamforming or beam steering radar system can be utilized to direct signals from individual antennas over a desired area or Field-of-View (FoV). For radar, this means the area within which the radar can detect objects, or targets. In wireless communications, this means the area within which a user (referred to as having User Equipment (UE)) is detected and a communication is maintained, such as to track a UE. In medical applications, the FoV is limited to an operational area within which procedures are performed. In accordance with various embodiments, the radar system can discern separation distances of individual beacons that are positioned in close proximity, i.e., at a separation distance of or less than, for example, about 1 mm, about 2 mm, about 3 mm, about 5 mm, about 10 mm, about 20 mm, or about 50 mm.

In accordance with various embodiments, the radar system disclosed herein can include one or more radar modules. One or more of the radar modules can be configured for transmitting a radio frequency signal at an initial frequency. The radar system can include a (first) beacon that can be configured to phase shift the initial frequency to a first frequency and another (second) beacon that can be configured to phase shift the initial frequency to a second frequency, where the second frequency is different from the first frequency. In some embodiments, the radar system can include an interface module that is configured for determining a physical distance between the first beacon and the second beacon based on a phase shift between the first frequency and the second frequency.

In some implementations, the interface module can be configured for use during a surgery, where the first beacon is disposed on a first anatomical portion of a body and the second beacon is disposed on a second anatomical portion of the body. The physical distance between the two beacons, thus locations of respective anatomical portions can be determined, and then used for determining a (separation) distance between the first and second anatomical portions of the body. The beacons can be implantable and/or encapsulated in a biocompatible shell. In various embodiments, the beacons are implantable and consumable beacons, e.g., disposable beacons. In various embodiments, the beacons are self-powered beacons. In various embodiments, the beacons can be color-coded beacon and/or can be implanted on the bone. In various embodiments, the beacons are preprogrammed for use as a certain frequency. In various embodiments, the beacons are active beacons for comprising circuitries, such as RFIC, for actively phase shifting, amplifying, etc.

In some implementations of the radar system, the radar system can also include a controller that is configured for interfacing the radar modules with the beacons. In such implementations, the initial frequency, the first frequency and the second frequency are determined by looking up at a predetermined lookup table stored within the controller, or any other components within the radar system.

In various embodiments, the controller can be configured to control operation of and receives radar signal from the one or more radar modules. In various embodiments, the controller has access to a memory storage, a look-up table, and to identify radar information and detected information to provide available information for visualization on surgical interface. In various embodiments, the radar system can be configured to interact with the beacons that shift the phase of a received radar transmission to generate a phase shifted response signal. Phase shifters are designed to assign specific frequency responses from one or more navigation modules to identify target locations, i.e., where the beacons are implanted or attached to.

In some implementations, a single radar module of the radar modules is configured for receiving the first frequency from the first beacon and the second frequency from the second beacon. In some implementations, multiple radar module each can be configured for receiving a specific frequency from a specific beacon. For example, a first radar module is configured for a first frequency from a first beacon, a second radar module is configured for a second frequency from a second beacon, and so on and so forth.

In various implementations, the radar modules can be positioned in a configuration for acquiring locations of the beacons via position triangulation. In such implementations, the physical distance between the beacons, for example, the first beacon and the second beacon, can be determined using both the locations of the first beacon and second beacon, and a phase shift between the first frequency and the second frequency.

As described in further detail below, the beacons (first and second, or any additional beacons utilized in the radar system) can comprise one or more of a receive antenna, a balun, an active phase shifter, a variable gain antenna, or a transmit antenna. Further, the active phase shifter used in such radar implementations can be configured to shift a received frequency based on the predetermined lookup table. In some embodiments, either of the first beacon or the second beacon can contain a radio frequency integrated circuit (RFIC) having a predetermined lookup table. For example, each value of a phase shift from the initial frequency to the first frequency or the second frequency (or any other frequencies) can be retrieved or determined based on the predetermined lookup table. As described below, such phase shifting from the initial frequency to the first frequency and the second frequency can be performed by the RFIC based on the predetermined lookup table.

In various implementations, the radar system can be configured for use in a surgical operating environment. The system can employ active tagging using one or more beacons that can be configured to operate at 77 GHz or any other frequencies. The radar system can achieve tagging and tracking capabilities via active phase shifting via an RFIC, with predetermined lookup table. As described herein, each or all of the radar modules can be configured to transmit and receive radar signals. In some implementations, one radar module may transmit while two radar modules may receive. In some implementations, all radar modules (e.g., a three radar modules) can transmit at the same time and all three radar modules receive.

In various embodiments, a computer configured for calculating a physical distance between the first beacon and the second beacon based on a phase shift between the first frequency and second frequency. In various embodiments, the one or more radar modules can be configured for receiving the first frequency and the second frequency, and for sending the phase shift between the first frequency and the second frequency to the controller.

In various embodiments, the controller can be configured to manage the one or more radar modules. In various embodiments, all three radar modules can be configured to transmit and/or receive RF signals. In various embodiments, one radar module can be configured to transmit the signal and two radar modules can be configured to receive the signal. In various embodiments, all the radar modules can transmit at the same time. In various embodiments, all the radar modules can be configured to receive the signal.

In various embodiments, the first beacon and the second beacon are implantable devices. In various embodiments, the first beacon and the second beacon are consumable beacons. In various embodiments, the first beacon and the second beacon are self-powered beacons. In various embodiments, the first beacon and the second beacon are active beacons that area configured for actively shifting phase and amplifying the signal. In various embodiments, the first beacon and the second beacon are color-coded beacons. In various embodiments, the first beacon and the second beacon are implantable on a bone of a person. In various embodiments, the first beacon and the second beacon are preprogrammed for operating within a predetermined frequency.

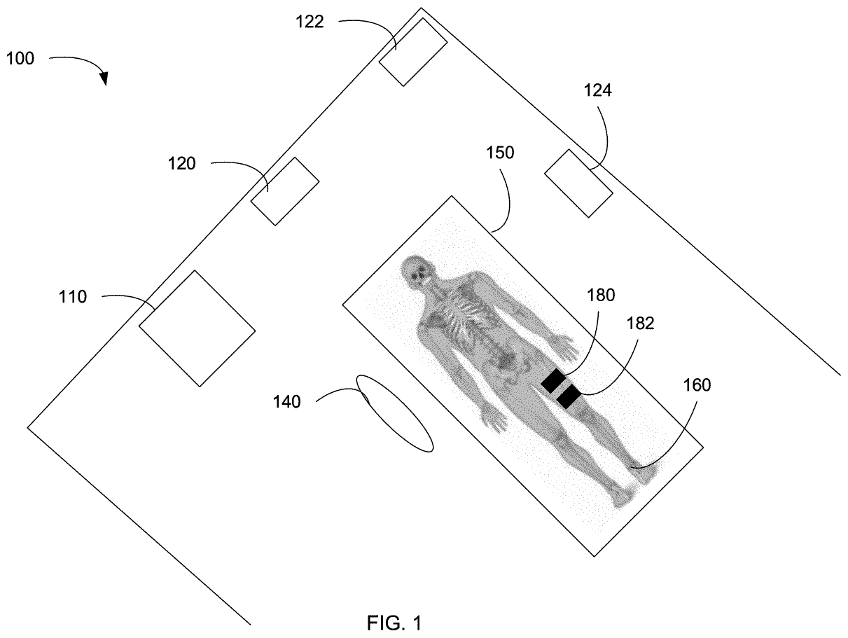

The following passages are described with respect to FIGS. 1-7, and represent non-limiting examples illustrated throughout the present disclosure.

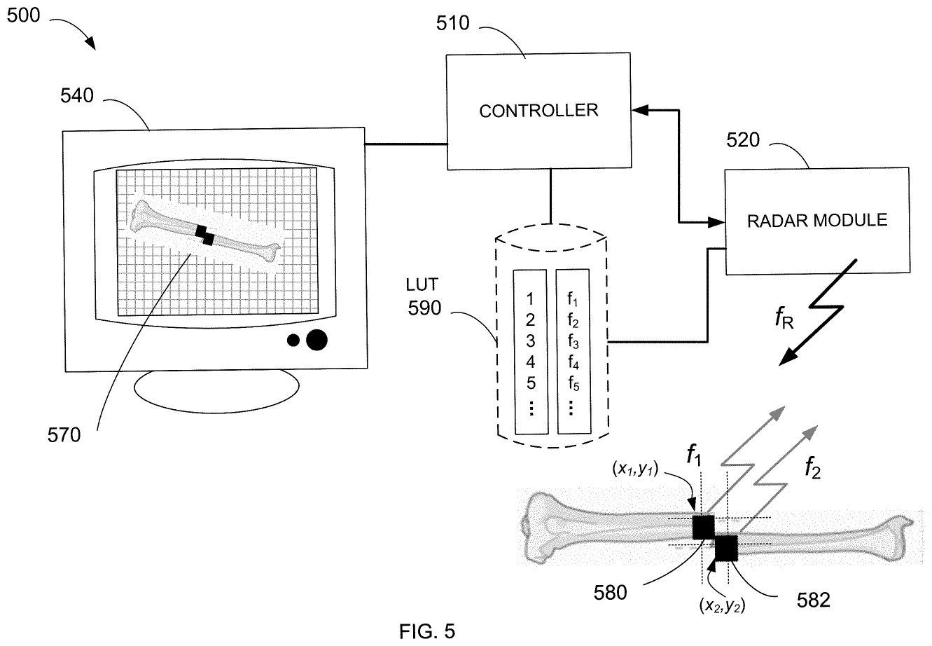

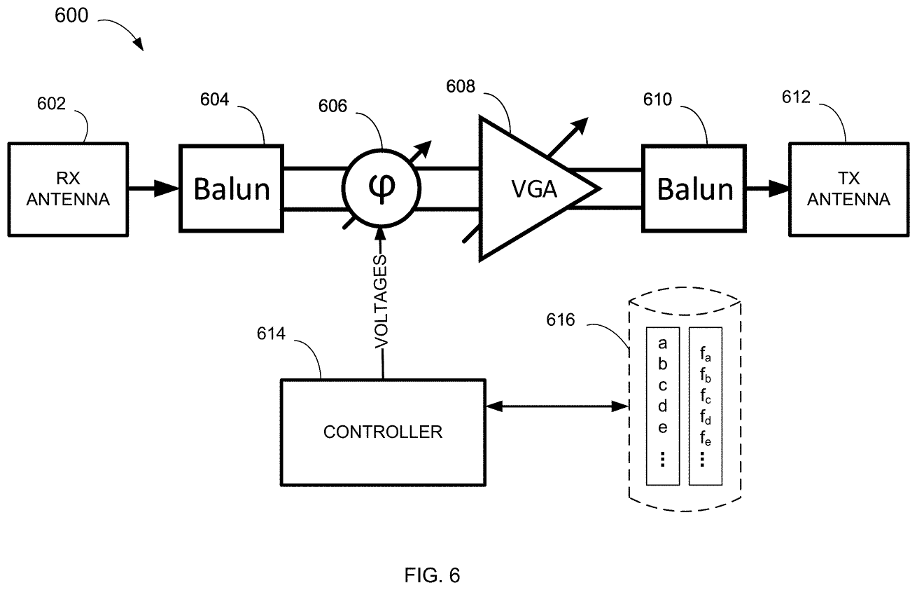

FIG. 1 illustrates an example deployment of a radar system 100 in a medical environment, in accordance with one or more implementations of the subject technology. As illustrated in FIG. 1, the medical environment, such as an operating room or surgical suite, includes a table 150 (e.g., an operating table) where a (patient) body 160 is positioned on the table 150. As shown in the figure, the radar system 100 includes radar modules 120, 122, and 124, a central controller 110 and beacons 180 and 182 to facilitate identification of specific locations on the body 160. The radar modules 120, 122, and 124 are configured to communicate with the beacons 180, 182 and are controlled by controller 110. Further details of controller 110 will be described with respect to FIGS. 5 and 6. Each of beacons 180 and 182 is a programmable unit controlled to respond to received radar signals with a specific frequency response. The response is used to identify the location of the corresponding beacons 180 and 182. In various embodiments, beacons 180 and 182 can be implantable and/or encapsulated in a biocompatible shell. In various embodiments, beacons 180 and 182 are consumable beacons, e.g., disposable beacons. In various embodiments, the beacons are self-powered beacons or color-coded. In various embodiments, beacons 180 and 182 are preprogrammed for use as a certain frequency. In various embodiments, beacons 180 and 182 are active beacons for comprising circuitries, such as RFIC, for actively phase shifting, amplifying, etc. The information is presented to a physician via a surgical interface 140. The radar system 100 is configured to provide detailed information to the physician to conduct a surgery or an operation with certainty based on precise locations determined using beacons 180 and 182.

In the medical environment illustrated in FIG. 1, the radar system 100 is configured to operate a beam steering radar which may be directed in over a range of angles in azimuth and elevation to detect and identify beacons 180 and 182, according to various implementations of the subject technology. One or more of the radar modules 120, 122, and 124 may be stationary or may be positioned along a track or other system to position and adjust as desired. Any number of radar modules may be implemented, and in some embodiments, a single unit may be used to cover a specific area. The radar system 100 can scan a Field of View (FoV) or specific area. As described in more detail below, the radar signal is transmitted according to a set of scan parameters that can be adjusted to result in multiple transmission beams. The radar modules 120, 122, and 124 transmit signals modulated according to a Frequency Modulated Continuous Wave (FMCW) as the change in frequency provides information about beacons 180 and 182, with each one implanted or attached to a specific portion of the body 160. An FMCW radar can transmit a sinusoidal signal at linearly increasing frequencies to generate a sawtooth wave when plotted as frequency over time and wherein one cycle is referred to a chirp. Each chirp has a start frequency, a bandwidth and a duration. The slope of the chirp defines the ramp rate of the signal. Other examples may use alternate modulation techniques and may incorporate different waveforms for the transmit signal. The scan parameters of radar modules 120, 122, and 124 may include, among others, the total angle of the scanned area defining the FoV, the beam width or the scan angle of each incremental transmission beam, the number of chirps in the radar signal, the chirp time, the chirp segment time, the chirp slope, and so on. The entire FoV or a portion of it can be scanned by a compilation of such transmission beams, which may be in successive adjacent scan positions or in a specific or random order. Note that the term FoV is used herein in reference to the radar transmissions and does not imply an optical FoV with unobstructed views. The scan parameters may also indicate the time interval between these incremental transmission beams, as well as start and stop angle positions for a full or partial scan.

The radar module 120, 122, and 124 transmits the FMCW signal, Tx; the transmitted signal, Tx, reflects off an object, referred to as a target (e.g., beacons 180 and 182), and the reflected signal or received signal, Rx, returns to the radar module 120, 122, and 124. Comparison of Tx and the corresponding Rx provides information about the physical distance from the radar module 120, 122, and 124 to beacons 180 and 182; this distance is referred to as the range. Various calculations of the signal information can provide more detailed information of beacons 180 and 182. This information may be used to identify the detected object, such as an anatomical portion of a person or vehicle, and parameters associated with the detected object. As the Rx signal is a delayed version of the Tx signal, the Rx signal and the Tx signal are mixed to form an instantaneous frequency (IF) which is the difference in the frequencies of the two signals. Range resolution refers to the ability of the radar module to resolve two closely spaced objects. In a given system if the objects are too close together they will appear as a single peak in the frequency spectrum. To distinguish the objects, the system is designed to increase the length of the IF signal, which increases proportionally with bandwidth. The greater the bandwidth, the greater the resolution will be in a system.

In the present examples, the radar modules 120, 122, and 124 operate to identify the location of the beacons 180, 182. In an example application, the radar modules 120, 122, and 124 each operate at unique frequencies. Consider transmissions, Tx, from radar module 124 at frequency f₁. When Tx is received at beacon 180, the reflected signal, Rx, returns to radar module 124 from which the target information is determined. In accordance with various embodiments, the beacon 180 can include phase shifting circuitry to change the frequency of the Tx signal such that the Rx signal received at the radar module 124 has a frequency shift used to recognize that the signal is returned from beacon 180 and identify the location of beacon 180. Similarly, beacon 182 may include phase shifting circuitry to change the frequency of an incident signal to frequency, f₂, which identifies beacon 182.

Although not illustrated explicitly in the medical environment of FIG. 1, other components, including for example, perception sensors, such as a camera or a lidar, may be useful in augmenting the object detection capabilities of the radar system 100. A camera or a lidar can be implemented in the radar system 100 to aid the radar modules 120, 122, and 124, and beacons 180 and 182, to detect visible objects and conditions, and to assist in providing auxiliary information to the physician. These may be used to enhance and improve the radar system 100 of FIG. 1. For example, a camera can be used to capture texture, color and contrast information at a high level of detail of a surgery scene in high resolution. A lidar may be used to further enhance the resolution and accuracy of the radar system 100 of FIG. 1.

To further enhance the radar system 100 of FIG. 1, a phase shift to a received signal can provide a change in the Doppler frequency measured and calculated at the one or more radar modules of the radar system 100. The Doppler effect is the apparent change in frequency when a beacon, e.g., beacon 180 moves toward or away from the radar transmitter, e.g., radar module 120. The apparent change in the frequency between the source and receiver is due to the relative motion between the source and receiver. This may be used to determine a speed and/or velocity of a detected target by a radar module. In the present system, the change in frequency is introduced by the beacon, or receiver, as an identifier. For example, the location of beacon 180 is thus determined by the range to beacon 180, the angle of arrival of the signal as determined by the radar modules 120, 122, and 124.

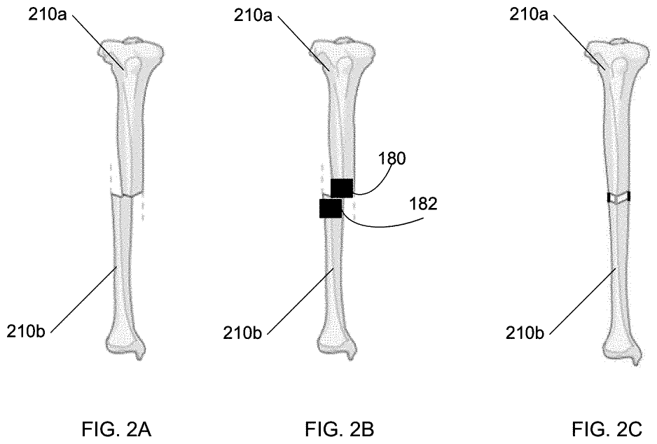

FIGS. 2A-2C illustrate an example anatomical situation wherein a radar system may be implemented in accordance with one or more implementations of the subject technology. As depicted in FIG. 2A, an example of a broken bone is illustrated as two individual pieces—portion 210a and portion 210b. To aid during surgery or operation of the bones, the radar system illustrated in FIG. 1 can be used for aligning or orienting of portion 210a and portion 210b. By using beacons 180 and 182 that are implanted or otherwise disposed at specific locations on the portion 210a and portion 210b of the bone as illustrated in FIG. 2B, the broken bone can be accurately reposition and realign using the radar system 100 of FIG. 1. By reading and monitoring locations, positions, angles, etc. of beacons 180 and 182, precise and proper alignment between the portion 210a and portion 210b of the bone can be ensured during the surgery to repair the broken bone. Such monitored information can be made available to a physician via a surgical interface or a computer user interface, which may provide visuals on a monitor, or on smart glasses for physician's use during the procedure, for example, through an augmented or virtual reality system. Once the surgery is completed and the portion 210a and portion 210b of the broken bone are reattached properly, beacons 180 and 182 can be removed as illustrated in FIG. 2C.

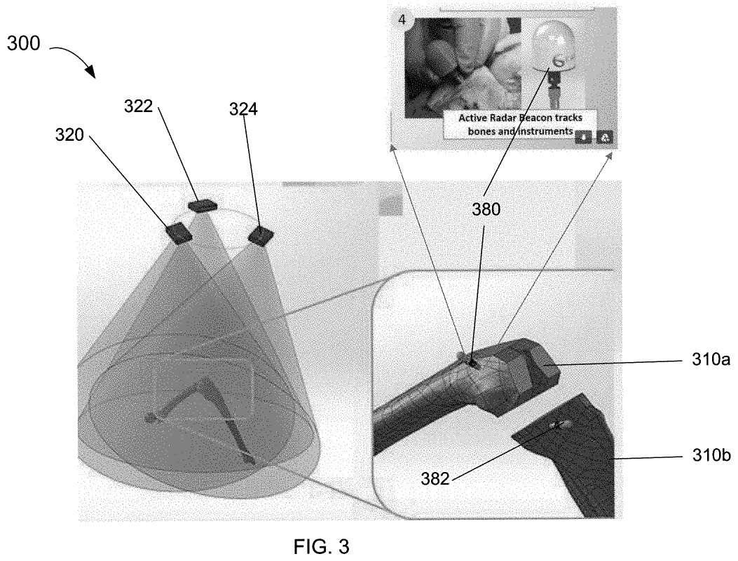

FIG. 3 illustrates an example implementation of a radar system 300, in accordance with one or more implementations of the subject technology. As illustrated in FIG. 3, the radar system 300 includes radar modules 320, 322, and 324 that are used to monitor and provide precise positions of beacons 380 and 382. The radar system 300 can be employed in another medical environment, for example, during surgery to repair a knee or an elbow joint, as illustrated in FIG. 3. By placing beacon 380 on a first portion 310a of the knee joint and beacon 382 on a second portion 310b of the knee joint, the radar system 300 can be used to monitor precise locations, angles, etc. of the beacons 380 and 382 during surgery to repair the knee. Similar to beacons 180 and 182, beacons 380 and 382 can be implantable and/or encapsulated in a biocompatible shell. In various embodiments, beacons 380 and 382 are consumable beacons, e.g., disposable beacons. In various embodiments, the beacons are self-powered beacons or color-coded. In various embodiments, beacons 380 and 382 are preprogrammed for use as a certain frequency. In various embodiments, beacons 380 and 382 are active beacons for comprising circuitries, such as RFIC, for actively phase shifting, amplifying, etc.

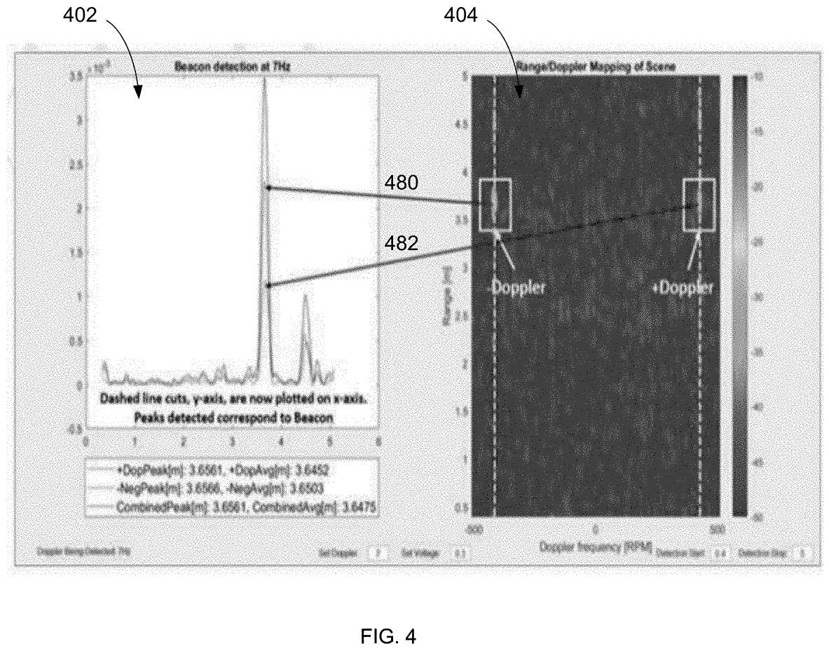

FIG. 4 illustrates example data acquired via a radar system, in accordance with one or more implementations of the subject technology. Plot 402 illustrated in FIG. 4 shows detection of radar signals from the beacons, such as beacons 180 and 182 or beacons 380 and 382 that are attached or implanted on different portions, e.g., portions 210a and 210b or portions 310a and 310b, of a bone or a knee joint, respectively. Here, the signals are overlapping and appearing at the same frequency range, although they differ in the amplitude of the signal. Using Doppler data plot 404 illustrate in FIG. 4, the two overlapping peaks can be distinguished as indicated by arrows 480 and 482. Using the combination of the two plots 402 and 404, the locations, positions, angles, etc. of beacons 180 and 182 or beacons 380 and 382 can be precisely monitored, tracked or otherwise used in the determination of the precise locations, positions, angles of the respective portions 210a and 210b or portions 310a and 310b during surgeries of the bone or the knee joint.

FIG. 5 illustrates a schematic diagram of a radar system 500 in accordance with one or more implementations of the subject technology. The radar system 500 includes a radar module 520 and beacons 580 and 582. The controller 510 controls operation of and receives information from radar module 520. The controller 510 has access to a memory storage, look up table (LUT) 590, to identify beacon information and detected information to provide this for visualization on surgical interface 540. The visualization 570 presented to the physician identifies the location of the navigation targets in completing the procedure. The radar system 500 enables the physician to operate on a small scale and with accuracy to improve outcomes. The surgical interface 540 may provide information to other modules, such as robotic instruments and other devices used to accomplish procedures. This information may provide location and alignment to improve alignment, which in the illustrated example is alignment of a broken bone.

Details in the LUT 590 identify each of the beacons 580 and 582 by response frequency. The radar module 520 receives signals at frequency f₁ from beacon 580 located as position (x₁,y₁) and provides this information to the controller 510. The radar module 520 receives signals at frequency f₁ from beacon 582 located as position (x₂,y₂) and provides this information to the controller 510. While both beacons 580 and 582 receive signals from radar module 520 at frequency f_{R}, however, each navigation target responds with a different, unique frequency response. The LUT 590 may be positioned with controller 510, distributed among one or more radar modules 520, as a separate module coupled to the radar system 500 or may be provided as input to the system such as by wireless signaling from a remote computing device.

In operation, the physician, technician or other professional will position beacon 580 and position (x₁,y₁) and beacon 582 and position (x₂,y₂). Each of beacons 580 and 582 is set, configured, programmed, or so forth to respond to radar signals with a response at a specific frequency that serves to identify the transmitting device, i.e. navigation target. The radar module 520 operates a given frequency, transmitting FMCW signals, and in response the navigation targets return the received signal at a different frequency, which achieved through phase shifting within the navigation target. The radar module 520 may generate a range-Doppler (RD) mapping for each received signal, and as beacons 580 and 582 each respond with a different frequency, they will be distinguished on the RD mapping. The RD mapping identifies the beacon and its location. The radar modules 520 may use LUT 590 to identify the beacon by frequency.

FIG. 6 illustrates components of an example beacon 600, in accordance with one or more implementations of the subject technology. As illustrated in FIG. 6, the beacon 600 includes a receive antenna 602, a balun 604, a phase shifter 606, a variable gain amplifier (VGA) 608, a balun 610 and a transmission antenna 612. As implemented, the receive antenna 602 is configured to receive a transmitted signal at an initial or incident frequency. The received signal is then transmitted to a phase shifter 606 via balun 604. The phase shifter 606 is a radio frequency integrated circuit (RFIC) that includes a look up table or have access to the controller 614, which has access to look up table (LUT) 616 to identify which frequency to shift to or an amount of required phase shift. In various implementations, the phase shifter module 606 can be a flip chip SiGe phase shifter with VGA chips enabled. The phase shifted signal is then transmitted from the phase shifter 606 to the VGA 608, which is then sent to the transmit antenna 612 via balun 610. The transmit antenna 612 then sends out the phase shifted signal at a different frequency that is different from the initial or incident frequency.

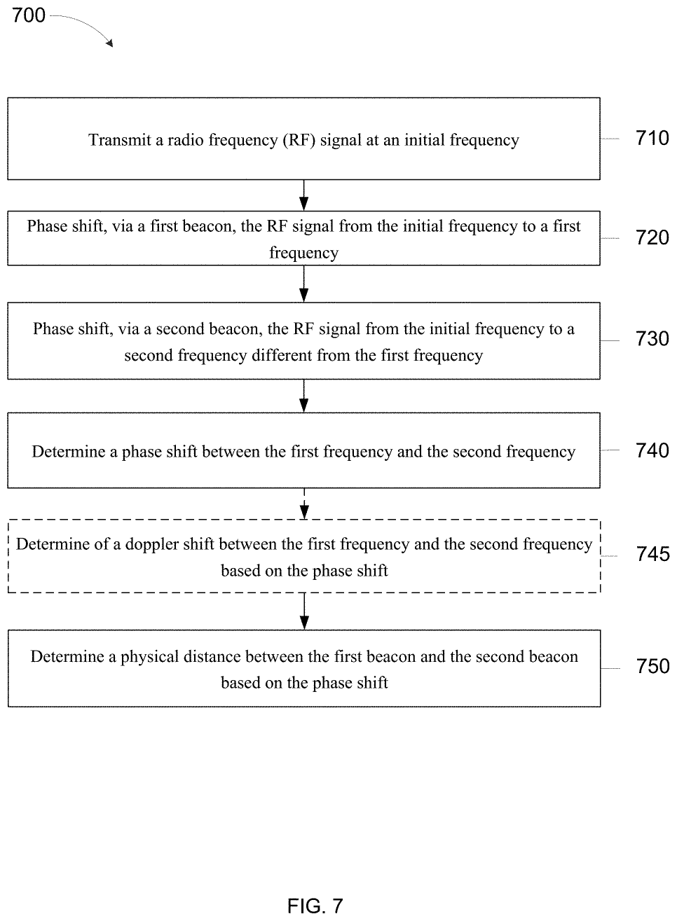

FIG. 7 illustrate a flowchart for a method 700 of using a radar system, in accordance with one or more implementations of the subject technology. The method 700 includes, at step 710, transmitting a radio frequency (RF) signal at an initial frequency. Using a radar system, such as the radar system 100 of FIG. 1 (or radar system 300 of FIG. 3), the RF signal can be transmitted from any of the radar modules 120, 122, or 124. The method 700 includes, at step 720, phase shifting, via a first beacon (e.g., beacons 180 or 380), the RF signal from the initial frequency to a first frequency, and at step 730, phase shifting, via a second beacon (e.g., beacons 182 or 382), the RF signal from the initial frequency to a second frequency different from the first frequency. The phase shifted RF signal at the first frequency and the phase shifted RF signal at the second frequency can be received by one or more radar modules of the radar system. The method 700 includes, at step 740, determining a phase shift between the first frequency and the second frequency. In some implementations, the method 700 optionally includes, at step 745, determining of a doppler shift between the first frequency and the second frequency based on the phase shift. The method 700 includes, at step 750, determining a physical distance between the first beacon and the second beacon based on the phase shift.

In various implementations, determining the physical distance between the first beacon and the second beacon can include determining a location of the first beacon and a location of the second beacon using position triangulation via the radar modules 120, 122, and 124 of the radar system 100. In various implementations, determining the physical distance between the first beacon and the second beacon can include using the location of the first beacon and the location of the second beacon and the calculated doppler shift to determine the physical distance.

In accordance with various embodiments, a radar system for use in a medical environment is provided. The radar system includes one or more radar modules configured for transmitting a radio frequency (RF) signal at an initial frequency, a first beacon configured for receiving the RF signal at the initial frequency and phase shifting the RF signal from the initial frequency to a first frequency, a second beacon configured for receiving the RF signal at the initial frequency and phase shifting the RF signal from the initial frequency to a second frequency different from the first frequency, and an interface module configured for determining a physical distance between the first beacon and the second beacon based on a phase shift between the first frequency and the second frequency.

In various embodiments, the radar system further includes a controller configured for interfacing the one or more radar modules with the first beacon and the second beacon. In various embodiments, the initial frequency, the first frequency and the second frequency are determined via a predetermined lookup table stored within the controller. In various embodiments, a single radar module from one or more radar modules is configured for receiving the RF signal at the first frequency from the first beacon and the RF signal at the second frequency from the second beacon. In various embodiments, one or more radar modules are positioned in a configuration for acquiring locations of the first beacon and the second beacon via position triangulation. In various embodiments, the physical distance between the first beacon and the second beacon is determined using both the locations of the first beacon and second beacon, and a phase shift between the first frequency and the second frequency.

In various embodiments, at least one of the first beacon and the second beacon includes a receive antenna, a balun, an active phase shifter, a variable gain antenna, and a transmit antenna, wherein the active phase shifter is configured to shift a received frequency of a signal based on the predetermined lookup table. In various embodiments, each of the first beacon and the second beacon comprises a radio frequency integrated circuit (RFIC) having a predetermined lookup table and each value of phase shift from the initial frequency to the first frequency and the second frequency is determined based on the predetermined lookup table. In various embodiments, the phase shifting is performed by the RFIC based on the predetermined lookup table.

In various embodiments, the interface module is configured for use during a surgery, wherein the first beacon is disposed on a first anatomical portion of a body and the second beacon is disposed on a second anatomical portion of the body, and the determined physical distance is used for determining a distance between the first and second anatomical portions of the body. In various embodiments, each of the first beacon and the second beacon is implantable and is encapsulated in a biocompatible shell.

In accordance with various embodiments, a method of using a radar system is provided. The method includes transmitting a radio frequency (RF) signal at an initial frequency; phase shifting, via a first beacon, the RF signal from the initial frequency to a first frequency; phase shifting, via a second beacon, the RF signal from the initial frequency to a second frequency different from the first frequency; determining a phase shift between the first frequency and the second frequency; and determining a physical distance between the first beacon and the second beacon based on the phase shift.

In various implementations, the method further includes, prior to determining the phase shift, receiving the RF signals at the first frequency and the second frequency at one or more radar modules of the radar system; and determining a doppler shift between the first frequency and the second frequency based on the phase shift. In various embodiments, determining the physical distance between the first beacon and the second beacon includes determining a first location of the first beacon and a second location of the second beacon using position triangulation via a plurality of radar modules disposed within a surgical room; and using the first location and the second location and the calculated doppler shift to determine the physical distance.

In various embodiments, each of the first beacon and the second beacon includes a radio frequency integrated circuit (RFIC) having a predetermined lookup table and each value of phase shift from the initial frequency to the first frequency and the second frequency is determined based on the predetermined lookup table. In various embodiments, the phase shifting is performed by the RFIC based on the predetermined lookup table.

In various embodiments, the first beacon is disposed on a first anatomical portion of a body and the second beacon is disposed on a second anatomical portion of the body. In various embodiments, the method further includes determining a distance between the first and second anatomical portions of the body based on the determined physical distance between the first beacon and the second beacon. In various embodiments, each of the first beacon and the second beacon is implantable and is encapsulated in a biocompatible shell.

In accordance with various embodiments, a distributed radar system for surgical operation is provided. The distributed radar system includes a radar system and an interface module. The radar system includes a first beacon disposed on a first anatomical portion of a body, a second beacon disposed on a second anatomical portion of the body, and a plurality of radar modules configured for position triangulation of the first beacon and the second beacon. At least one radar module in the plurality of radar modules is configured for transmitting a radio frequency (RF) signal at an initial frequency. The first beacon is configured for phase shifting the RF signal from the initial frequency to a first frequency and the second beacon is configured for phase shifting the RF signal from the initial frequency to a second frequency different from the first frequency. The interface module is configured for determining a phase shift between the first frequency and second frequency.

In various embodiments, locations of the first beacon and second beacon are determined via the position triangulation with the plurality of radar modules. In various embodiments, a physical distance between the first anatomical portion and the second anatomical portion is determined using the phase shift between the first frequency and the second frequency, and the locations of the first beacon and second beacon.

It is appreciated that the previous description of the disclosed examples is provided to enable any person skilled in the art to make or use the present disclosure. Various modifications to these examples will be readily apparent to those skilled in the art, and the generic principles defined herein may be applied to other examples without departing from the spirit or scope of the disclosure. Thus, the present disclosure is not intended to be limited to the examples shown herein but is to be accorded the widest scope consistent with the principles and novel features disclosed herein.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While this specification contains many specifics, these should not be construed as limitations on the scope of what may be claimed, but rather as descriptions of particular implementations of the subject matter. Certain features that are described in this specification in the context of separate implementations can also be implemented in combination in a single implementation. Conversely, various features that are described in the context of a single implementation can also be implemented in multiple implementations separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. Moreover, the separation of various system components in the aspects described above should not be understood as requiring such separation in all aspects, and it should be understood that the described program components and systems can generally be integrated together in a single hardware product or packaged into multiple hardware products. Other variations are within the scope of the following claim.

What is claimed is:

1. A radar system, comprising:
one or more radar modules configured for transmitting a radio frequency (RF) signal at an initial frequency;
a first beacon configured for receiving the RF signal at the initial frequency and phase shifting the RF signal from the initial frequency to a first frequency;
a second beacon configured for receiving the RF signal at the initial frequency and phase shifting the RF signal from the initial frequency to a second frequency different from the first frequency; and
an interface module configured for determining a physical distance between the first beacon and the second beacon based on a phase shift between the first frequency and the second frequency.

2. The radar system of claim 1, further comprising:
a controller configured for interfacing the one or more radar modules with the first beacon and the second beacon, wherein the initial frequency, the first frequency and the second frequency are determined via a predetermined lookup table stored within the controller.

3. The radar system of claim 1, wherein a single radar module from the one or more radar modules is configured for receiving the RF signal at the first frequency from the first beacon and the RF signal at the second frequency from the second beacon.

4. The radar system of claim 1, wherein the one or more radar modules are positioned in a configuration for acquiring locations of the first beacon and the second beacon via position triangulation.

5. The radar system of claim 4, wherein the physical distance between the first beacon and the second beacon is determined using both the locations of the first beacon and second beacon, and a phase shift between the first frequency and the second frequency.

6. The radar system of claim 1, wherein at least one of the first beacon and the second beacon comprises a receive antenna, a balun, an active phase shifter, a variable gain antenna, and a transmit antenna, wherein the active phase shifter is configured to shift a received frequency of a signal based on the predetermined lookup table.

7. The radar system of claim 1, wherein each of the first beacon and the second beacon comprises a radio frequency integrated circuit (RFIC) having a predetermined lookup table and each value of phase shift from the initial frequency to the first frequency and the second frequency is determined based on the predetermined lookup table.

8. The radar system of claim 7, wherein the phase shifting is performed by the RFIC based on the predetermined lookup table.

9. The radar system of claim 1, wherein the interface module is configured for use during a surgery, wherein the first beacon is disposed on a first anatomical portion of a body and the second beacon is disposed on a second anatomical portion of the body, and the determined physical distance is used for determining a distance between the first and second anatomical portions of the body.

10. The radar system of claim 9, wherein each of the first beacon and the second beacon is implantable and is encapsulated in a biocompatible shell.

11. A method of using a radar system, comprising:
transmitting a radio frequency (RF) signal at an initial frequency;
phase shifting, via a first beacon, the RF signal from the initial frequency to a first frequency;
phase shifting, via a second beacon, the RF signal from the initial frequency to a second frequency different from the first frequency;
determining a phase shift between the first frequency and the second frequency; and
determining a physical distance between the first beacon and the second beacon based on the phase shift.

12. The method of claim 11, further comprising:
prior to determining the phase shift, receiving the RF signals at the first frequency and the second frequency at one or more radar modules of the radar system; and
determining a doppler shift between the first frequency and the second frequency based on the phase shift.

13. The method of claim 12, wherein determining the physical distance between the first beacon and the second beacon comprises:
determining a first location of the first beacon and a second location of the second beacon using position triangulation via a plurality of radar modules disposed within a surgical room; and
using the first location and the second location and the calculated doppler shift to determine the physical distance.

14. The method of claim 11, wherein each of the first beacon and the second beacon comprises a radio frequency integrated circuit (RFIC) having a predetermined lookup table and each value of phase shift from the initial frequency to the first frequency and the second frequency is determined based on the predetermined lookup table.

15. The method of claim 14, wherein the phase shifting is performed by the RFIC based on the predetermined lookup table.

16. The method of claim 11, wherein the first beacon is disposed on a first anatomical portion of a body and the second beacon is disposed on a second anatomical portion of the body, the method further comprising:
determining a distance between the first and second anatomical portions of the body based on the determined physical distance between the first beacon and the second beacon.

17. The method of claim 16, wherein each of the first beacon and the second beacon is implantable and is encapsulated in a biocompatible shell.

18. A distributed radar system for surgical operation, comprising:
a radar system comprising:
a first beacon disposed on a first anatomical portion of a body;
a second beacon disposed on a second anatomical portion of the body;
a plurality of radar modules configured for position triangulation of the first beacon and the second beacon,
wherein at least one radar module in the plurality of radar modules is configured for transmitting a radio frequency (RF) signal at an initial frequency,
wherein the first beacon is configured for phase shifting the RF signal from the initial frequency to a first frequency and the second beacon is configured for phase shifting the RF signal from the initial frequency to a second frequency different from the first frequency;
an interface module configured for determining a phase shift between the first frequency and second frequency.

19. The distributed radar system of claim 18, wherein locations of the first beacon and second beacon are determined via the position triangulation with the plurality of radar modules.

20. The distributed radar system of claim 18, wherein a physical distance between the first anatomical portion and the second anatomical portion is determined using the phase shift between the first frequency and the second frequency, and the locations of the first beacon and second beacon.
